(19) United States
(12) Patent Application Publication
(10) Pub. No.: US 2015/0057320 A1
(43) Pub. Date: Feb. 26, 2015

(54) TRANSTHYRETIN LIGANDS CAPABLE OF INHIBITING RETINOL-DEPENDENT RBP4-TTR INTERACTION FOR TREATMENT OF AGE-RELATED MACULAR DEGENERATION, STARGARDT DISEASE, AND OTHER RETINAL DISEASE CHARACTERIZED BY EXCESSIVE LIPOFUSCIN ACCUMULATION

(71) Applicant: Konstantin PETRUKHIN, New Windsor, NY (US)

(72) Inventor: Konstantin PETRUKHIN, New Windsor, NY (US)

(73) Assignee: The Trustees of Columbia University in the City of New York, New York, NY (US)

(21) Appl. No.: 14/530,516

(22) Filed: Oct. 31, 2014

Related U.S. Application Data
(63) Continuation-in-part of application No. PCT/US2013/038910, filed on Apr. 30, 2013.
(60) Provisional application No. 61/641,124, filed on May 1, 2012.

(51) Int. Cl. A61K 31/423; A61K 31/05; A61K 31/196; A61K 31/343

(52) U.S. Cl. CPC A61K 31/423; A61K 31/343; A61K 31/05; A61K 31/196
USPC 514/375; 514/469; 514/734; 514/567

(57) ABSTRACT

A method for treating a disease characterized by excessive lipofuscin accumulation in the retina of a mammal afflicted therewith comprising administering to the mammal an effective amount of a transthyretin (TTR) ligand.

This application is a continuation-in-part of and claims benefit of PCT International Application No. PCT/US2013/038910, filed Apr. 30, 2013, which claims the benefit of U.S. Provisional Application No. 61/641,124, filed May 1, 2012, the contents of each of which are hereby incorporated by reference in their entirety.

Throughout this application, certain publications are referenced in parenthesis. Full citations for these publications may be found immediately preceding the claims. The disclosures of these publications in their entireties are hereby incorporated by reference into this application in order to describe more fully the state of the art to which this invention relates.

This invention was made with government support under grant numbers NS067594 and NS074476 awarded by the National Institutes of Health. The government has certain rights in the invention.

### BACKGROUND OF THE INVENTION

Age-related macular degeneration (AMD) is the leading cause of blindness in developed countries. It is estimated that 62.9 million individuals worldwide have the most prevalent atrophic (dry) form of AMD; 8 million of them are Americans. Due to increasing life expectancy and current demographics this number is expected to triple by 2020. There is no FDA-approved treatment for dry AMD. Given the lack of treatment and high prevalence, development of drugs for dry AMD is of upmost importance.

Clinically, atrophic AMD represents a slowly progressing neurodegenerative disorder in which specialized neurons (rod and cone photoreceptors) die in the central part of the retina called macula [1]. Histopathological and clinical imaging studies indicate that photoreceptor degeneration in dry AMD is triggered by abnormalities in the retinal pigment epithelium (ROE) that lies beneath photoreceptors and provides critical metabolic support to these light-sensing neuronal cells.

Experimental and clinical data indicate that excessive accumulation of cytotoxic autofluorescent lipidprotein-retinoid aggregates (lipofuscin) in the RPE is a major trigger of dry AMD [2-7]. Excessive accumulation of lipofuscin is also a critical feature of autosomal recessive Stargardt's disease (STGD), an untreatable form of inherited macular dystrophy caused by genetic mutations in the ABCA4 gene. STGD is one of the most prevalent causes of juvenile and early adult vision loss and, although representing an orphan disease, presents a major public health problem. The major cytotoxic component of RPE lipofuscin in dry AMD and STGD is pyridinium bisretinoid A2E (FIG. 1).

A2E is a product of condensation of all-trans retinaldehyde with phosphatidylethanolamine which occurs in the retina in a non-enzymatic manner and, as illustrated in FIG. 2, can be considered a by-product of a properly functioning visual cycle [8]. Light-induced isomerization of 11-cis retinaldehyde to its all-trans form is the first step in a signaling cascade that mediates light perception. The visual cycle is a chain of biochemical reactions that regenerate visual pigment (11-cis retinaldehyde conjugated to opsin) following exposure to light.

As cytotoxic A2E is formed during the course of a normally functioning visual cycle, it has been suggested that partial pharmacological inhibition of the visual cycle may represent a treatment strategy for dry AMD and other disorders characterized by excessive accumulation of lipofuscin, such as STGD [9-12]. As rates of the visual cycle and A2E production in the retina depend on the influx of all-trans retinol from serum to the RPE (FIG. 2), it has been suggested that partial pharmacological downregulation of serum retinol may represent a target area in dry AMD treatment [13].

Tafamidis [55], a potent TTR kinetic stabilizer, is the most clinically advanced TTR ligand. It has been approved by EMEA (EU regulatory agency) but rejected by the FDA as a treatment for Transthyretin Familial Amyloid Polyneuropathy (TTR-FAP) [56]. Tafamidis is not available in the US neither as a reagent nor as the FDA-approved drug. We synthesized tafamidis and conducted its characterization in a number of in vitro and in vivo assays.

### SUMMARY OF THE INVENTION

The inventions provides a method for treating a disease characterized by excessive lipofuscin accumulation in the retina of a mammal afflicted therewith comprising administering to the mammal an effective amount of a transthyretin (TTR) ligand.

### BRIEF DESCRIPTION OF THE FIGURES

FIG. 1. Structure of bisretinoid A2E, a cytotoxic component of retinal lipofuscin.

FIG. 2. Visual cycle and biosynthesis of A2E. A2E biosynthesis begins when a portion of all-trans-retinal escapes the visual cycle (yellow box) and non-enzymatically reacts with phosphatidyl-ethanolamine forming the A2E precursor, A2-PE. The absence of a functional ABCA4 (Stargardt's disease) increases the likelihood of bisretinoid formation. Uptake of serum retinol to the RPE (gray box) fuels the cycle (From ref. 1).

FIG. 3. Three-dimensional structure of the RBP4-TTR-retinol complex. Tetrameic TTR is shown in blue, light blue, green and yellow. RBP is shown in red and retinol is shown in gray (from ref. [14]). A TTR tetramer contains two thyroxine-binding pockets (central cavity; two binding sites per TTR tetramer) which are not occupied by thyroxine in 90% of TTR tetramers [15].

FIG. 4. TTR amyloidogenesis cascade (from ref [26]). Foramyloidogenesis to occur, the unliganded TTR tetramer must first dissociate into four folded monomers and undergo partial denaturation. These pieces then subsequently misassemble into a variety of aggregate structures including toxic amyloid fibrils. Complexation with retinol-RBP4 or binding of natural or synthetic TTR ligands stabilizes TTR tetramers and prevents amyloidogenesis.

FIG. 5. Schematic depiction of the HTRF-based assay format for identification of desired TTR ligands. RBP4-TTR interaction induced by saturating concentrations of retinol will induce FRET signal that can be reduced by TTR ligands allosterically antagonizing retinol-dependent RBP4-TTR interaction.

FIG. 6. Dose titrations of all-trans retinol in the HTRF-based RBP4-TTR interaction assay.

FIG. 7. Dose titrations of A1120 (panel A) and fenretinide (panel B) in the presence of high concentrations of all-trans retinol in the HTRF based RBP4-TTR interaction assay.

FIG. 8. Analysis of 446 compounds from the NIH Clinical Collection in the HTRF based RBP4-TTR interaction assay. Positive control wells (yellow circles) contain 20 uM A1120 and 4.5 μM retinal; negative control wells (blue triangles) contain 4.5 μM retinol and DMSO in place of an antagonist; test compound wells (red circles) contain compounds at 10 μM along with 4.5 μM retinol. Data from wells with DMSO only are shown as green circles. For normalization of data from 6 plates HTRF signal from retinal wells was assumed to be 100%; signal from DMSO wells is assumed to be 0%.

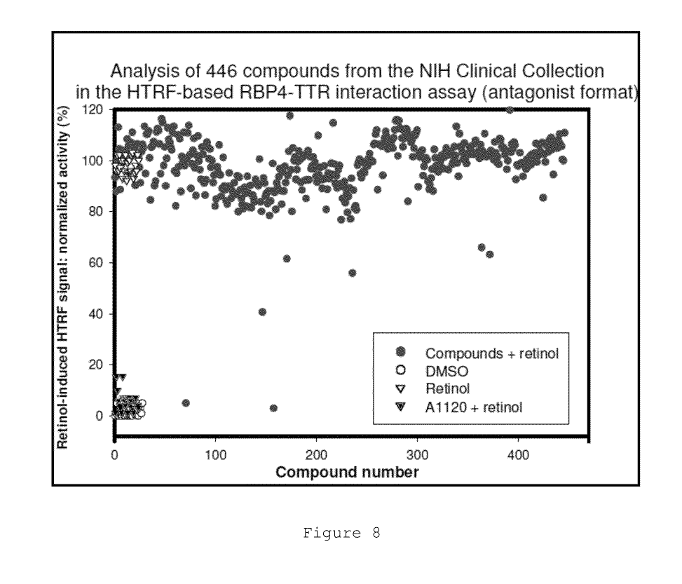

FIG. 9. Dose-dependent inhibition of the retinol induced RBP4-TTR interaction in the HTRF assay for compounds identified in the Biomol/Enzo library of nuclear receptor ligands (Right panel) and in the NIH Clinical Collection (Center and Left panels). A1120 (left panel, brown line) is included as a positive control.

FIG. 10. Dose-dependent inhibition of the retinol-induced RBP4-TTR interaction in the HTRF assay for compounds identified in the NIH Clinical Collection. S-FTA, a weak RBP4 ligand is included as a positive control.

FIG. 11. Saturation binding of 3H-Resveratrol to TTR. Specific binding was calculated by subtracting non-specific binding measured in the presence of 100 μM.

FIG. 12. Dose titration analysis of Tiagabine-HCl, Nifedipine, and Benzbromarone in the TTR binding assay.

FIG. 13. Analysis of test compounds in the TTR aggregation assay. Purified TTR was incubated with compounds at pH 4.4. followed by glutaraldehyde cross-linking and analysis of the protein complex using SDS-PAGE. The left lane contains the protein cross-linked at neutral pH. The right lane contains the cross-linked complex incubated at pH 4.4 without compounds. Test cpd X and Test cpd Y are compounds unrelated to the compounds discussed in this section.

FIG. 14. Reduction in Serum RBP4 in response to compound administration to wild-type mice.

FIG. 15. HTRF-based assay and TTR binding assay for additional compounds.

FIG. 16. Alternate schematic depiction of the HTRF-based assay format for identification of desired TTR ligands. RBP4-TTR interaction induced by saturating concentrations of retinol will induce FRET signal that can be reduced by TTR ligands allosterically antagonizing retinol-dependent RBP4-TTR interaction.

FIG. 17. Dose titrations of tafamidis along with the positive control, A1120, in the HTRF-based RBP4-TTR interaction assay.

FIG. 18. Dose titration analysis of tafamidis, benzbromarone, mefenamic acid, resveratrol and CU163 (unrelated compound) in the FP-based TTR binding assay. Structure of the fluorescence-polarization TTR probe used in the binding assay is shown as an inset.

FIG. 19. Reduction in serum RBP4 induced by a single dose administration of tafamidis in mice.

FIG. 20. Reduction in serum RBP4 in response to chronic tafamidis treatment.

### DETAILED DESCRIPTION OF THE INVENTION

The inventions provides a method for treating a disease characterized by excessive lipofuscin accumulation in the retina of a mammal afflicted therewith comprising administering to the mammal an effective amount of a transthyretin (TTR) ligand.

In some embodiments, the disease is further characterized by bisretinoid-mediated macular degeneration.

In some embodiments, the TTR ligand is an allosteric antagonist of retinol dependent RBP4-TTR interaction.

In some embodiments, the TTR ligand stabilizes the tetrameric structure of TTR.

In some embodiments, the amount of the ligand is effective to lower serum concentration of RBP4 in the mammal.

In some embodiments, the amount of the ligand is effective to lower the retinal concentration of a bisretinoid in lipofuscin in the mammal.

In some embodiments, the disease is further characterized by bisretinoid-mediated macular degeneration.

In some embodiments, the bisretinoid is A2E. In some embodiments the bisretinoid is isoA2E. In some embodiments the bisretinoid is A2-DHP-PE. In some embodiments the bisretinoid is atRAL di-PE.

In some embodiments, bisretinoid-mediated macular degeneration may be Age-Related Macular Degeneration or Stargardt Disease.

In some embodiments, the bisretinoid-mediated macular degeneration is Age-Related Macular Degeneration.

In some embodiments, the bisretinoid-mediated macular degeneration is dry (atrophic) Age-Related Macular Degeneration.

In some embodiments, the bisretinoid-mediated macular degeneration is Stargardt Disease.

In some embodiments, the bisretinoid-mediated macular degeneration is Best disease.

In some embodiments, the bisretinoid-mediated macular degeneration is adult vitelliform maculopathy.

In some embodiments, the bisretinoid-mediated macular degeneration is Stargardt-like macular dystrophy.

In some embodiments, the disease characterized by excessive lipofuscin accumulation in the retina may be Age-Related Macular Degeneration or Stargardt Disease.

In some embodiments, the disease characterized by excessive lipofuscin accumulation in the retina is Age-Related Macular Degeneration.

In some embodiments, the disease characterized by excessive lipofuscin accumulation in the retina is dry (atrophic) Age-Related Macular Degeneration.

In some embodiments, the disease characterized by excessive lipofuscin accumulation in the retina is Stargardt Disease.

In some embodiments, the disease characterized by excessive lipofuscin accumulation in the retina is Best disease.

In some embodiments, the disease characterized by excessive lipofuscin accumulation in the retina is adult vitelliform maculopathy.

In some embodiments, the disease characterized by excessive lipofuscin accumulation in the retina is Stargardt-like macular dystrophy.

The bisretinoid-mediated macular degeneration may comprise the accumulation of lipofuscin deposits in the retinal pigment epithelium.

In some embodiments, the TTR ligand is benzbromarone, resveratrol, mefenamic acid, tafamidis, flufenamic acid, diflunisal, diclofenac or flurbiprofen.

In some embodiments, a method for treating an ocular disease in a mammal afflicted therewith comprising administering to the mammal an effective amount of a transthyretin (TTR) ligand.

In some embodiments of the above method, the TTR ligand is benzbromarone, resveratrol, mefenamic acid, tafamidis, flufenamic acid, diflunisal, diclofenac or flurbiprofen.

In some embodiments, the ocular disease is not characterized by excessive lipofuscin accumulation in the retina.

In some embodiments, the mammal does not have excessive lipofuscin accumulation in the retina.

In some embodiment, the ocular disease is diabetic retinopathy, light-induced photoreceptor degeneration, or retinal detachment.

In some embodiments of the above method, the TTR ligand reduces the level of retinoids in the mammal.

In some embodiments of the above method, the TTR ligand reduces the level of visual cycle retinoids in the mammal.

In some embodiments of the above method, the TTR ligand modulates the visual cycle in the mammal.

In some embodiments, a method for treating diabetes in a mammal afflicted therewith comprising administering to the mammal an effective amount of a transthyretin (TTR) ligand.

In some embodiments, a method for treating insulin resistance in a mammal afflicted therewith comprising administering to the mammal an effective amount of a transthyretin (TTR) ligand.

In some embodiments of the above method, the TTR ligand is benzbromarone, resveratrol, mefenamic acid, tafamidis, flufenamic acid, diflunisal, diclofenac or flurbiprofen.

In some embodiments of the above method, the diabetes is type-2 diabetes.

In some embodiments, a method for treating obesity in a mammal afflicted therewith comprising administering to the mammal an effective amount of a transthyretin (TTR) ligand.

In some embodiments of the above method, the TTR ligand is benzbromarone, resveratrol, mefenamic acid, tafamidis, flufenamic acid, diflunisal, diclofenac or flurbiprofen.

In some embodiments of the above method, the TTR ligand down-regulates RPB4 in the mammal.

In some embodiments of the above method, the TTR ligand reduces the level of RPB4 in the mammal.

As used herein, “TTR ligand” is intended to mean a moiety that interacts with transthyretin.

As used herein, a “ligand” refers to a molecule or compound or entity that interacts with a ligand binding site, including substrates or analogues or parts thereof. As described herein, the term “ligand” may refer to compounds that bind to the protein of interest. A ligand may be an agonist, an antagonist, or a modulator. Or, a ligand may not have a biological effect. Or, a ligand may block the binding of other ligands thereby inhibiting a biological effect. Ligands may include, but are not limited to, small molecule inhibitors. These small molecules may include peptides, peptidomimetics, organic compounds and the like. Ligands may also include polypeptides and/or proteins.

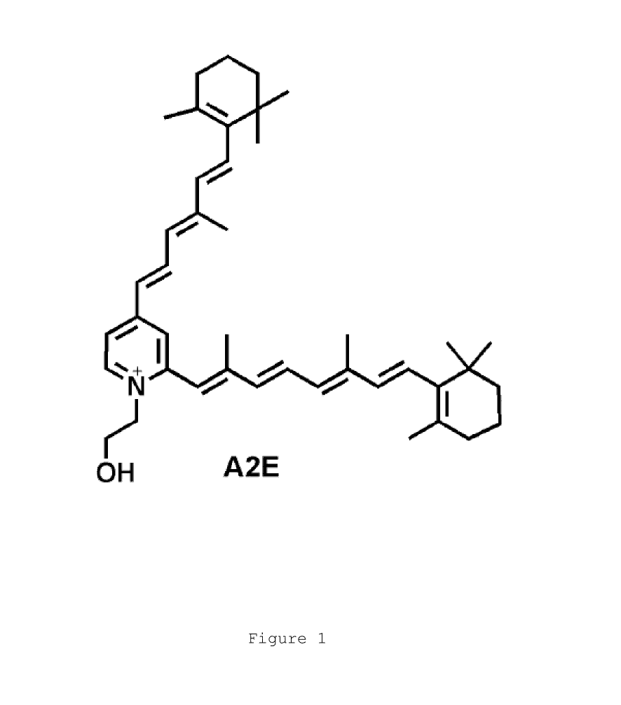

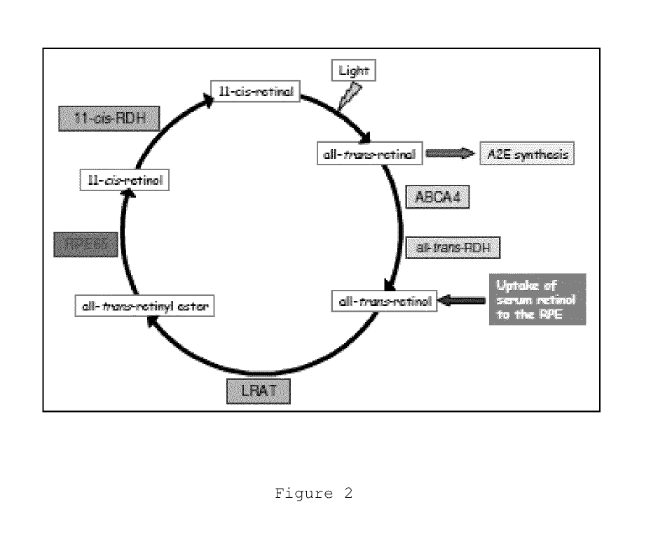

As used herein, “bisretinoid lipofuscin” is lipofuscin containing a cytotoxic bisretinoid. Cytotoxic bisretinoids include but are not necessarily limited to A2E, isoA2E, atRAL di-PE, and A2-DHP-PE (FIG. 1-3).

As used herein, “allosteric antagonist of retinol dependent RBP4-TTR interaction” is intended to mean an antagonist that inhibits retinol dependent RBP4-TTR interaction without binding to the retinol-binding pocket in the RBP4.

As used herein, the description “pharmaceutically active” is used to characterize a substance, compound, or composition suitable for administration to a subject and furnishes biological activity or other direct effect in the treatment, cure, mitigation, diagnosis, or prevention of disease, or affects the structure or any function of the subject. Pharmaceutically active agents include, but are not limited to, substances and compounds described in the Physicians' Desk Reference (PDR Network, LLC; 64th edition; Nov. 15, 2009) and “Approved Drug Products with Therapeutic Equivalence Evaluations” (U.S. Department of Health and Human Services, 30^{th} edition, 2010), which are hereby incorporated by reference.

Another aspect of the invention comprises a compound used in the method of the present invention as a pharmaceutical composition.

The compounds used in the method of the present invention may may be in a salt form. As used herein, a “salt” is a salt of the instant compound which has been modified by making acid or base salts of the compounds. In the case of the use of the compounds for treatment of bisretinoid-mediated macular degeneration, the salt is pharmaceutically acceptable. Examples of pharmaceutically acceptable salts include, but are not limited to, mineral or organic acid salts of basic residues such as amines. The term “pharmaceutically acceptable salt” in this respect, refers to the relatively non-toxic, inorganic and organic base addition salts of the compounds. These salts can be prepared in situ during the final isolation and purification of the compounds, or by separately reacting purified compounds in their free acid form with a suitable organic or inorganic base, and isolating the salt thus formed.

As used herein, “treating” means slowing, stopping, or preventing the progression of a disease. An embodiment of “treating bisretinoid-mediated macular degeneration” is delaying or preventing the onset, progression, or mitigating severity of vision loss.

The compounds used in the method of the present invention may may be administered in various forms, including those detailed herein. The treatment with the compound may be a component of a combination therapy or an adjunct therapy, i.e. the mammal in need of the drug is treated or given another drug for the disease in conjunction with the compounds used in the method of the present invention. This combination therapy can be sequential therapy where the mammal is treated first with one drug and then the other or the two drugs are given simultaneously. These can be administered independently by the same route or by two or more different routes of administration depending on the dosage forms employed.

As used herein, a “pharmaceutically acceptable carrier” is a pharmaceutically acceptable solvent, suspending agent or vehicle, for delivering the instant compounds to the mammal. The carrier may be liquid or solid and is selected with the planned manner of administration in mind. Liposomes are also a pharmaceutically acceptable carrier.

The dosage of the compounds administered in treatment will vary depending upon factors such as the pharmacodynamic characteristics of the compound and its mode and route of administration; the age, sex, metabolic rate, absorptive efficiency, health and weight of the recipient; the nature and extent of the symptoms; the kind of concurrent treatment being administered; the frequency of treatment with; and the desired therapeutic effect.

A dosage unit of the compounds used in the method of the present invention may comprise the compound alone, or mixtures of the compound with additional compounds used to treat lipofuscin-mediated macular degeneration. The compounds can be administered in oral dosage forms as tablets, capsules, pills, powders, granules, elixirs, tinctures, suspensions, syrups, and emulsions. The compounds may also be administered in intravenous (bolus or infusion), intraperitoneal, subcutaneous, or intramuscular form, or introduced directly, e.g. by injection or other methods, into the eye, all using dosage forms well known to those of ordinary skill in the pharmaceutical arts.

The compounds used in the method of the present invention can be administered in a mixture with suitable pharmaceutical diluents, extenders, excipients, or carriers (collectively referred to herein as a pharmaceutically acceptable carrier) suitably selected with respect to the intended form of administration and as consistent with conventional pharmaceutical practices. The unit will be in a form suitable for oral, rectal, topical, intravenous or direct injection or parenteral administration. The compounds can be administered alone but are generally mixed with a pharmaceutically acceptable carrier. This carrier can be a solid or liquid, and the type of carrier is generally chosen based on the type of administration being used. In one embodiment the carrier can be a monoclonal antibody. The active agent can be co-administered in the form of a tablet or capsule, liposome, as an agglomerated powder or in a liquid form. Examples of suitable solid carriers include lactose, sucrose, gelatin and agar. Capsule or tablets can be easily formulated and can be made easy to swallow or chew; other solid forms include granules, and bulk powders. Tablets may contain suitable binders, lubricants, diluents, disintegrating agents, coloring agents, flavoring agents, flow-inducing agents, and melting agents. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents. Oral dosage forms optionally contain flavorants and coloring agents. Parenteral and intravenous forms may also include minerals and other materials to make them compatible with the type of injection or delivery system chosen.

Specific examples of pharmaceutical acceptable carriers and excipients that may be used to formulate oral dosage forms of the present invention are described in U.S. Pat. No. 3,903,297, issued Sep. 2, 1975. Techniques and compositions for making dosage forms useful in the present invention are described-in the following references: 7 Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modem Pharmaceutics Drugs and the Pharmaceutical Sciences, Vol 40 (Gilbert S. Banker, Christopher T. Rhodes, Eds.). All of the aforementioned publications are incorporated by reference herein.

Tablets may contain suitable binders, lubricants, disintegrating agents, coloring agents, flavoring agents, flow-inducing agents, and melting agents. For instance, for oral administration in the dosage unit form of a tablet or capsule, the active drug component can be combined with an oral, non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol and the like. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

The compounds used in the method of the present invention can also be administered in the form of liposome delivery systems, such as small unilamellar vesicles, large unilamallar vesicles, and multilamellar vesicles. Liposomes can be formed from a variety of phospholipids, such as cholesterol, stearylamine, or phosphatidylcholines. The compounds may be administered as components of tissue-targeted emulsions.

The compounds used in the method of the present invention may also be coupled to soluble polymers as targetable drug carriers or as a prodrug. Such polymers include polyvinylpyrrolidone, pyran copolymer, polyhydroxylpropylmethacrylamide-phenol, polyhydroxyethylasparta-midephenol, or polyethyleneoxide-polylysine substituted with palmitoyl residues. Furthermore, Compound 1 may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels.

The compounds used in the method of the present invention can be administered orally in solid dosage forms, such as capsules, tablets, and powders, or in liquid dosage forms, such as elixirs, syrups, and suspensions. It can also be administered parentally, in sterile liquid dosage forms.

Gelatin capsules may contain the compounds used in the method of the present invention and powdered carriers, such as lactose, starch, cellulose derivatives, magnesium stearate, stearic acid, and the like. Similar diluents can be used to make compressed tablets. Both tablets and capsules can be manufactured as immediate release products or as sustained release products to provide for continuous release of medication over a period of hours. Compressed tablets can be sugar coated or film coated to mask any unpleasant taste and protect the tablet from the atmosphere, or enteric coated for selective disintegration in the gastrointestinal tract.

For oral administration in liquid dosage form, the compounds used in the method of the present invention may be combined with any oral, non-toxic, pharmaceutically acceptable inert carrier such as ethanol, glycerol, water, and the like. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents.

Liquid dosage forms for oral administration can contain coloring and flavoring to increase patient acceptance. In general, water, a suitable oil, saline, aqueous dextrose (glucose), and related sugar solutions and glycols such as propylene glycol or polyethylene glycols are suitable carriers for parenteral solutions. Solutions for parenteral administration preferably contain a water soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol. Suitable pharmaceutical carriers are described in Remington's Pharmaceutical Sciences, Mack Publishing Company, a standard reference text in this field.

The compounds used in the method of the present invention may also be administered in intranasal form via use of suitable intranasal vehicles, or via transdermal routes, using those forms of transdermal skin patches well known to those of ordinary skill in that art. To be administered in the form of a transdermal delivery system, the dosage administration will generally be continuous rather than intermittent throughout the dosage regimen.

Parenteral and intravenous forms may also include minerals and other materials to make them compatible with the type of injection or delivery system chosen.

The compounds used in the method of the present invention and compositions thereof of the invention can be coated onto stents for temporary or permanent implantation into the cardiovascular system of a subject.

The compounds and compositions of the present invention are useful for the prevention and treatment of lipofuscin-mediated macular degeneration.

Except where otherwise specified, when the structure of a compound of this invention includes an asymmetric carbon atom, it is understood that the compound occurs as a racemate, racemic mixture, and isolated single enantiomer. All such isomeric forms of these compounds are expressly included in this invention. Except where otherwise specified, each stereogenic carbon may be of the R or S configuration. It is to be understood accordingly that the isomers arising from such asymmetry (e.g., all enantiomers and diastereomers) are included within the scope of this invention, unless indicated otherwise. Such isomers can be obtained in substantially pure form by classical separation techniques and by stereochemically controlled synthesis, such as those described in “Enantiomers, Racemates and Resolutions” by J. Jacques, A. Collet and S. Wilen, Pub. John Wiley & Sons, NY, 1981. For example, the resolution may be carried out by preparative chromatography on a chiral column.

The subject invention is also intended to include all isotopes of atoms occurring on the compounds disclosed herein. Isotopes include those atoms having the same atomic number but different mass numbers. By way of general example and without limitation, isotopes of hydrogen include tritium and deuterium. Isotopes of carbon include C-13 and C-14.

It will be noted that any notation of a carbon in structures throughout this application, when used without further notation, are intended to represent all isotopes of carbon, such as ¹²C, ¹³C, or ¹⁴C. Furthermore, any compounds containing ¹³C or ¹⁴C may specifically have the structure of any of the compounds disclosed herein.

The compounds used in the method of the present invention may be prepared by techniques well know in organic synthesis and familiar to a practitioner ordinarily skilled in the art. However, these may not be the only means by which to synthesize or obtain the desired compounds.

The compounds used in the method of the present invention may be prepared by techniques described in Vogel's Textbook of Practical Organic Chemistry, A. I. Vogel, A. R. Tatchell, B. S. Furnis, A. J. Hannaford, P. W. G. Smith, (Prentice Hall) 5^{th} Edition (1996), March's Advanced Organic Chemistry: Reactions, Mechanisms, and Structure, Michael B. Smith, Jerry March, (Wiley-Interscience) 5th Edition (2007), and references therein, which are incorporated by reference herein. However, these may not be the only means by which to synthesize or obtain the desired compounds.

The compounds and/or ligands used in the method of the present invention may be purchased from a variety of chemical suppliers. Benzbromarone (Catalog No. B5774), resveratrol (Catalog No. R5010), mefenamic acid (Catalog No. M4267), flufenamic acid (Catalog No. F9005), diflunisal (Catalog No. D3281), diclofenac (Catalog No. D6899) and flurbiprofen (Catalog No. F8514) are available from Sigma-Aldrich (St. Louis, Mo., USA).

It will also be noted that any notation of a hydrogen in structures throughout this application, when used without further notation, are intended to represent all isotopes of hydrogen, such as ¹H, ²H, or ³H. Furthermore, any compounds containing ²H or ³H may specifically have the structure of any of the compounds disclosed herein.

Isotopically-labeled compounds can generally be prepared by conventional techniques known to those skilled in the art using appropriate isotopically-labeled reagents in place of the non-labeled reagents employed.

Each embodiment disclosed herein is contemplated as being applicable to each of the other disclosed embodiments. Thus, all combinations of the various elements described herein are within the scope of the invention.

This invention will be better understood by reference to the Examples which follow, but those skilled in the art will readily appreciate that the specific experiments detailed are only illustrative of the invention as described more fully in the claims which follow thereafter.

### EXAMPLES

### Example 1

### TR-FRET Assay for Allosteric Antagonists of Retinol-Induced RBP4-TTR Interaction

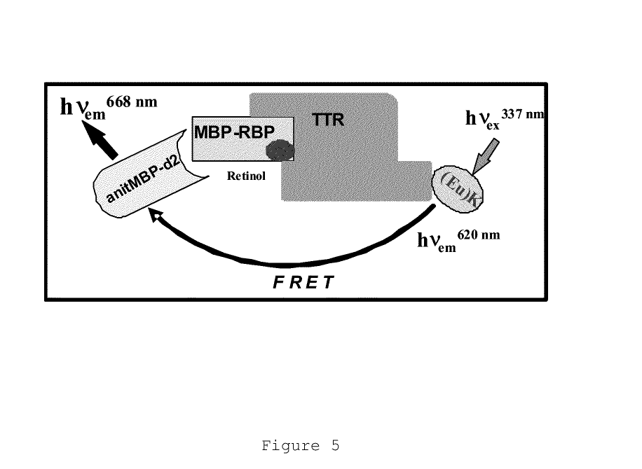

TRFRET (Time-Resolved Fluorescence Resonance Energy Transfer) is an assay format widely used in characterization of compounds affecting protein-protein interactions [32-34]. HTRF (Homogeneous Time-Resolved Fluorescence) variant of TR-FRET is the most advanced as it has improved light capturing due to the use of Eu3+ cryptates. In the presence of retinol, RBP4-TTR interaction induces FRET that can be registered as increased ratio of 668/620 fluorescence signals (FIG. 5).

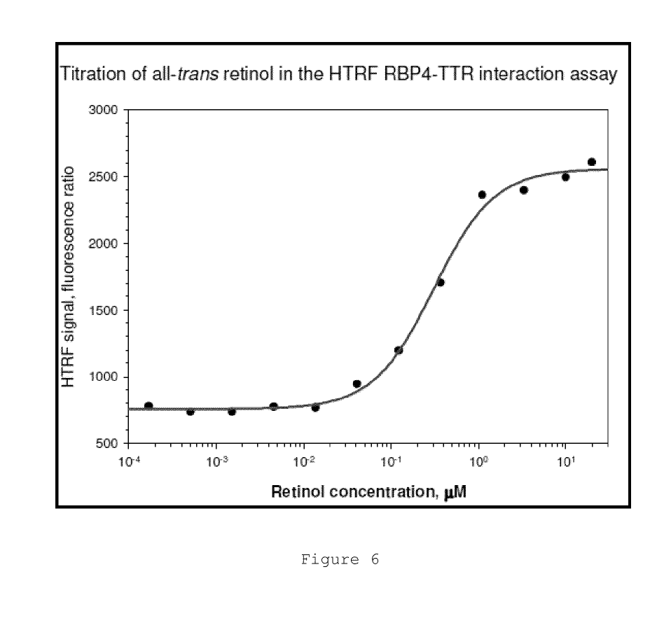

Retinol-dependent RBP4-TTR interaction can be inhibited by RBP4 ligands which competitively antagonize retinol binding to RBP4 [9, 10]. However, if the assay is conducted at saturating retinol concentrations, the screen will allow identification of allosteric antagonists of retinol-dependent RBP4-TTR interaction. Synthetic TTR ligands are primary candidates for being such allosteric antagonists as the TTR tetramer in a retinol-RBP4-TTR complex contains two unoccupied well-defined ligand binding pockets for thyroxine located in the proximity to the RBP4-TTR interaction interface (FIG. 1). Binding of a desired TTR ligands would disrupt RBP4-TTR interaction induced by saturating concentrations of retinol which will be registered as a decrease in FRET signal (FIG. 5). An assay was developed using E. coli expressed MBP-tagged RBP4 and commercially available TTR labeled directly with Eu3+ cryptate. In addition to MBP-RBP4 and Eu3+ (K)-TTR, a detector reagent anti-MBP-d2 was present in the reaction mix. The assay was first optimized in the agonist mode; sensitivity and dynamic range of the assay was optimized in respect to RBP4, TTR and detection reagent concentrations. In order to determine the optimum concentration of all-trans retinol stimulating the RBP4-TTR interaction we performed a 12-point retinol titration (FIG. 6). It was demonstrated that all-trans retinal stimulates RBP4-TTR interaction in a dose dependent manner (FIG. 6) with EC₅₀ of 308 nM.

Given that there are no known TTR ligands capable of antagonizing retinol dependent RBP4-TTR interaction, no TTR-specific positive control could be used in assay development. At the same time, a highly potent RBP4 antagonist, A1120, capable of disrupting retinol-dependent RBP4-TTR interaction with Ki of 8.3 nM has been recently described [16]. The assay was converted to the antagonist mode by testing concentrations of retinol within the 1-10 μM range and using 40 μM concentration of A1120. During conversion of the assay to the antagonist mode significant consideration was given to the range of appropriate agonist (retinol) concentrations. Retinol concentration has to be high enough in order to allow preferential identification of allosteric antagonists acting independent of retinol binding to RBP4. At the same time, A1120, a direct retinol antagonist, was used as a positive control in the assay characterization. The compromise retinol concentration in the antagonist mode in regard of the assay sensitivity and dynamic range was found to be in the 4.5-6.5 μM range which is appropriately higher than EC₅₀=308 nM for retinol (FIG. 6). In order to formally establish S/B, % CV values for the antagonist format and to calculate a Z-score, several independent test runs were performed with up to 20 identical negative control wells (wells containing 4.5 uM retinol along with up to 20 identical positive control wells (wells containing 4.5 μM retinol plus 40 μM A1120). In addition, the assay was run in the presence of 0.1-1.0% DMSO in order to assess DMSO tolerability.

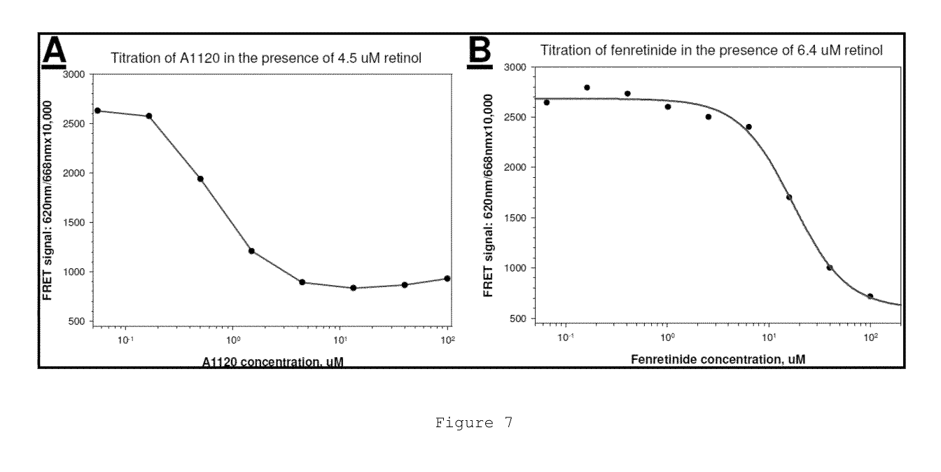

Overall, the allosteric antagonism format of the assay was optimized for low-volume 384-well plates with the final volume of 16 μl. To additionally document assay performance in the antagonist mode, titrations of A1120 and fenretinide, two direct retinol antagonists, were conducted in the presence of high concentrations of retinol (FIG. 7).

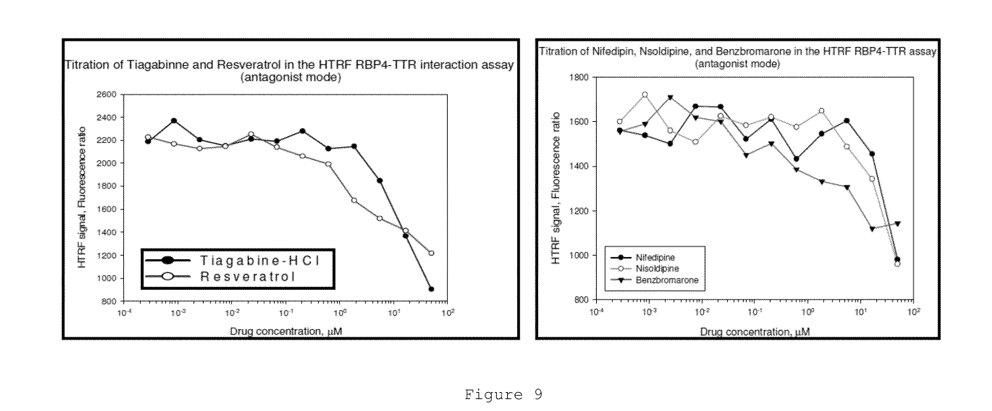

As expected, due to high concentration of retinol required for preferential identification of allosteric inhibitors of retinol-dependent RBP4-TTR interaction, high concentrations of direct retinol antagonists, A1120 and fenretinide, were required for inhibition of the RBP4-TTR interaction in assay conditions (IC₅₀=2.2 μM for A1120 and 17.3 μM for fenretinide). These results confirmed that A1120 can be used as a positive control during assay implementation. In order to prove that the developed assay is suitable for identification and characterization of TTR ligands allosterically inhibiting retinol-dependent RBP4-TTR interaction, this assay was used to screen a commercially available NIH Clinical Collection (446 diverse compounds with a history of use in human clinical trials) where a set of 7 compounds (includes Tiagabine-HCl, Resveratrol, Nifedipine, Benzbromarone, and Nisoldipine) were shown to exhibit greater than 30% inhibition of the retinol-induced HTRF signal in the RBP4-TTR interaction assay (FIG. 9). The screen of the compound collection demonstrated assay stability in regard of the plate-to-plate and day-to-day variations with the stable assay window of 3-4-fold (calculated with the use of A1120 as a positive control) and a Z-score of 0.67.

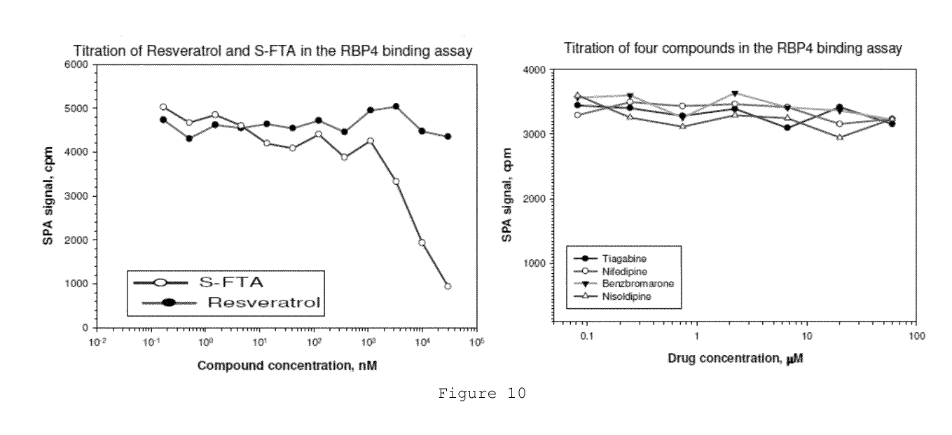

Five positives from the NIH Clinical collection library (Tiagabine-HCl, Resvratrol, Nifedipine, Benzbromarone, and Nisoldipine) were titrated in the primary assay along with the positive control, A1120, in order to confirm that they dose-dependently antagonize retinol-induced RBP4-TTR interaction (FIG. 10).

Given that at least one compound identified in the NIH clinical collection, resveratrol, is a known high affinity TTR ligand with proven ability to bind to TTR tetramers [25], it was reasonable to suggest that the HTS-compatible assay we developed is suitable for identification of TTR ligands capable of antagonizing retinol-dependent RBP4-TTR interaction. However, this assay could not determine with certainty that identified compounds are TTR-specific as potent direct retinol antagonists binding to RBP4, such as A1120 and fenretinide, would also induce the decrease of the HTRF signal in the primary assay (FIG. 7).

To be able to define specificity of compounds identified in the future HTS two additional follow-up assays required for determining compound specificity were implemented: SPA-based RBP4 binding assay (counterscreen) and filtration-based binding assay for TTR.

### Example 2

### RBP4 Binding Assay

Scintillation proximity assay (SPA) is a versatile platform suitable for development of binding assays for the variety of targets. Given the homogeneous nature of this format, SPA assays are fully compatible with HTS requirements and suitable for post-HTS evaluation of putative hits. The high specific activity radioligand required for the development of SPA binding assay for RBP4, [11, 12-³H(N)]-Retinol with 48.7 Ci/mmol, is commercially available.

For assay implementation, human untagged RBP4 purified from urine of a tubular protienuria patient (commercially available from Fitzgerald industries) was biotinylated and Streptavidin-PVT SPA beads from PerkinElmer were used. Assay conditions were optimized in a 96-well format for reduction of nonspecific binding, non-proximity effects, temperature, incubation time and in regard of the radioligand and RBP4 concentrations. Non-radioactive retinol was used as a competitor in assay optimization and characterization. For conducting compound analysis the [11, 12-³H(N)]-Retinol concentration was fixed at 10 nM and the biotinylated RBP4 concentration was 25 nM. In order to formally establish S/B, % CV values and to calculate a Z-score, we performed several independent test runs with up to 20 identical negative control wells (wells containing radioligand only) along with 20 identical positive control wells (wells containing radioligand plus 20 μM cold retinol). The assay demonstrated the exceptional 7-10 fold window with a Z score of higher than 0.7. We utilized the SPA assay to analyze 5 positive hits identified in the pilot screen. Following titrations of retinol and A1120, it was confirmed that our experimental Kd values were in line with those that were previously reported for these two compounds [13, 16, 35].

The results showed that the five compounds (Tiagabine-HCl, Resvratrol, Nifedipine, Benzbromarone, and Nisoldipine) did not display the RBP4 binding activity which is consistent with a notion that they may antagonize retinol-dependent RBP4-TTR interaction independent of binding to the retinol-binding pocket in the RBP4. As some of these compounds may be TTR ligands, a TTR binding assay was developed to assess this possibility.

### Example 3

### Transthyretin Filtration-Based Binding Assay

Transthyretin is a tetrameric protein with two clearly defined thyroxin-binding pockets [36]. Numerous publications report the design of the competition binding assays for TTR that utilize [¹²⁵I]-thyroxine as a radioligand [37-39]. Additionally, a synthesis of the FITC modified TTR ligand that can be used in a fluorescence polarization (FP)-based binding assay has been recently reported [31]. Unfortunately, the FP ligand is not available commercially and its multistep synthesis [31] requires significant investments.

In order to definitively prove that a subset of compounds identified in the primary screen represent TTR ligands a TTR binding assay that utilizes 3H-resveratrol, an established TTR ligand, was developed [24, 25, 40]. For assay implementation, untagged TTR preparation purified from human plasma (commercially available from Calbiochem) and [1,3-benzenediol-2 3H]-Resveratrol, 18.6 Ci/mmol, available from Perkin Elmer, were used. Non-radioactive resveratrol was used as a competitor in assay optimization and characterization. The assay was conducted in a 96-well format. To separate the bound radioligand, gel-filtration chromatography on 96-well Spin Desalting plates (Thermo Scientific) was used.

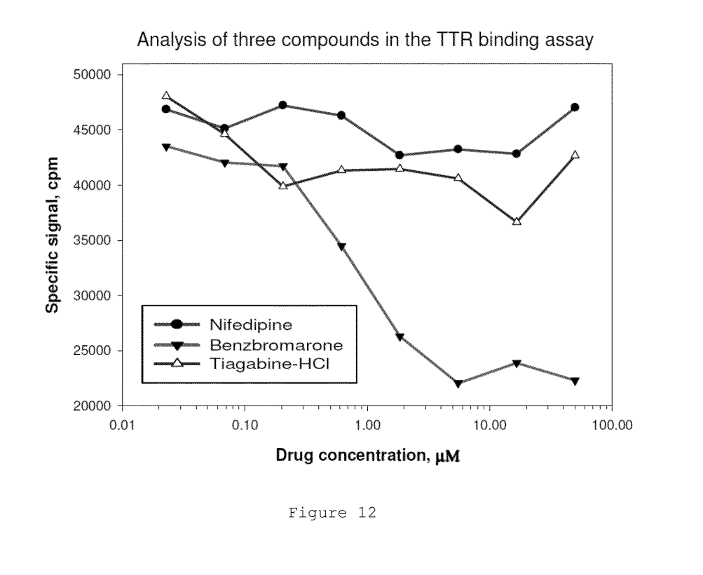

After conducting saturation binding experiments (exemplified in FIG. 12) direct Resveratrol binding to TTR was confirmed. Based on the saturation curve for [1,3-benzenediol-2 3H]-Resveratrol, a concentration of the radioligand was fixed at 3 μM during competitive binding experiments in which four remaining compounds suspected to be TTR ligands capable of antagonizing retinol-dependent RBP4-TTR interaction were analyzed.

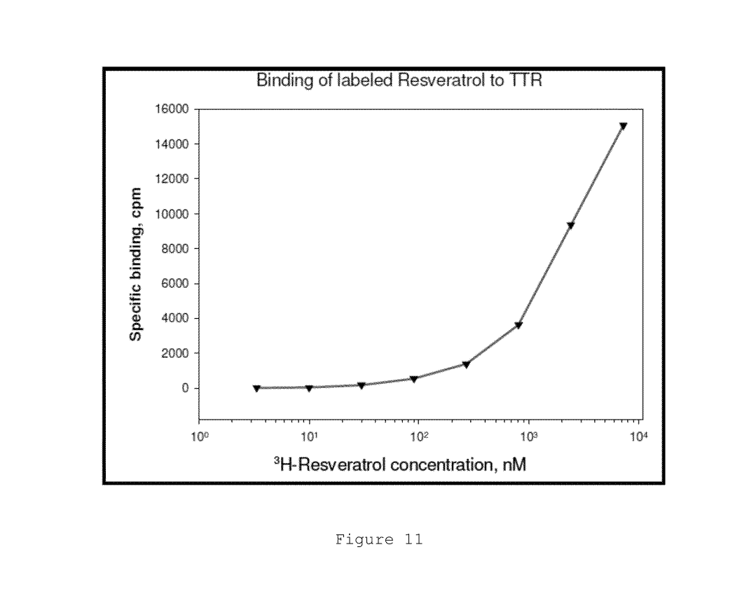

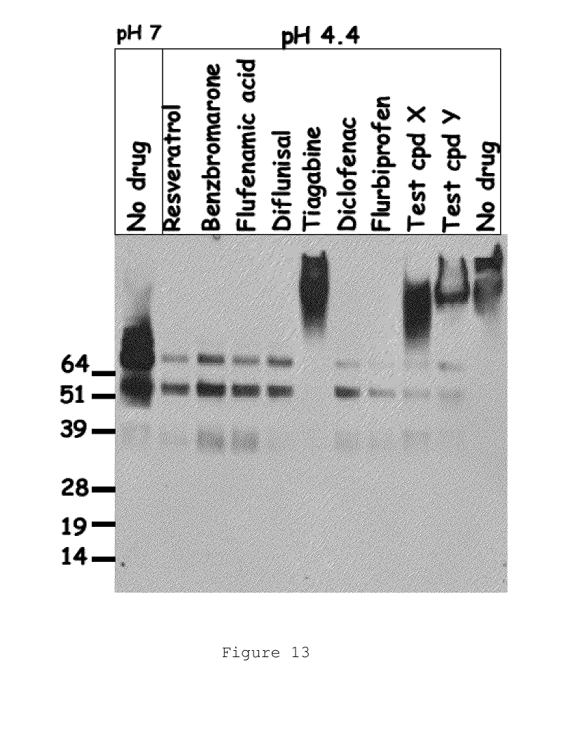

Along with Resveratrol (FIG. 12), only Benzbromarone (FIG. 13) was definitively shown to bind to TTR as can be judged by displacement of radioactive resveratrol (FIG. 13, blue curve). To our knowledge, Benzbromarone was not known before to be a TTR ligand. Tiagabine-HCl and Nifedipine did not bind to TTR (nor did they bind to RBP4: FIG. 11, Right panel) indicating that they may represent the artifacts of the primary screen. Benzbromarone and Resveratrol activity in a battery of in vitro assays proves the existence of bona fide TTR ligands capable of antagonizing retinol-dependent RBP4-TTR interaction.

### Example 4

### Gel-Based TTR Fibril Formation Assay

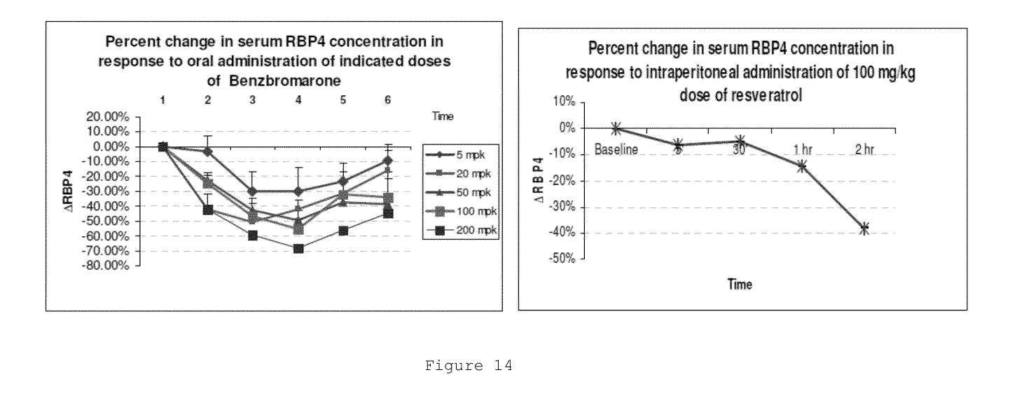

One of the desired attributes for TTR ligands capable of antagonizing retinol-dependent RBP4-TTR interaction would be stabilization of TTR tetramers so such compounds can be used in patients who, along with dry AMD and STGD, carry proamyloidogenic mutations within the TTR gene. Acidic pH-induced TTR fibril formation is an in vitro assay widely used for assessment of compounds capable of stabilizing TTR tetramers [30, 41, 42]. To investigate whether the two identified TTR ligands, resveratrol and benzbromarone, which disrupt retinolinduced RBP4-TTR interaction, can also stabilize TTR tetramers under amyloid forming (acidic) conditions previously described acidic pH-induced TTR fibril formation assay was established [41]. Included in the analysis were NSAID compounds that are known to bind to TTR and stabilize TTR tetramers [24, 25]. Test compounds were incubated with purified TTR in the sodium acetate buffer at pH 4.4 followed by glutaraldehyde cross-linking, neutralization and analysis of the cross-linked complex in SDS-PAGE. Purified TTR exists in solution as a tetramer which when cross-linked is visualized in SDS-PAGE as a 56 kDa band (FIG. 14, Left lane). Acidic pH induces TTR fibril formation and generation of aggregates which, when cross-linked, are visualized as higher molecular weight complexes in SDS-PAGE (FIG. 14, Right lane). Compounds binding to TTR and stabilizing its tetrameric structure prevent the formation of high molecular weight aggregates in this assay.

Both verified positive hits from the primary assay, resveratrol and benzbromarone, were capable of preventing TTR fibril formation and aggregation in assay conditions (FIG. 14). This proves the existence of TTR ligands capable of antagonizing retinol-dependent RBP4-TTR interaction while stabilizing the TTR tetrameric structure. Interestingly, tiagabine, a false positive from the primary screen (no binding to TTR, FIG. 13), did not protect TTR from aggregation (FIG. 14). Additional compounds such as flufenamic acid, diclofenac, diflunisal, flurbiprofen, that were reported in the literature to bind to TTR and stabilize its tetramers [24, 25] were shown to inhibit TTR aggregation in our experiments (FIG. 14) confirming the performance of our in vitro fibril formation assay.

### Example 5

### Characterization of In Vivo Activity for Two TTR Ligands Capable of Antagonizing Retinol-Dependent RBP4-TTR Interaction

Rates of the visual cycle and bisretinoid production in the retina depend on the influx of all-trans retinol from serum to the RPE. RPE retinol uptake depends on serum RBP4 concentrations. In order to determine whether the two TTR ligands capable of antagonizing retinol-dependent RBP4-TTR interaction in vitro can reduce serum RBP4 levels in vivo, dosing in wild type mice was conducted and assessed the reduction in serum RBP4 in response to compound administration. For oral gavage administration benzbromarone was formulated in 1% methylcellulose, 1% Tween 80. For IP administration, resveratrol was dissolved in a minimum volume of alcohol. For benzbromarone dosing, blood samples were collected from a tail vein before dosing and at 5 min, 30 min, 1 hr, 2 hr, 4 hr, and 6 hr timepoints. For resveratrol dosing blood samples were collected from a tail vein before dosing and at 5 min, 30 min, 1 hr, and 2 hr. Whole blood was drawn into a centrifuge tube and was let clot at room temperature for 30 min followed by centrifugation at 2,000×g for 15 minutes at +4° C. to collect serum. Serum RBP4 was measured using the RBP4 (mouse/rat) dual ELISA kit (Enzo Life Sciences) following the manufacturer's instructions. This data confirm the effect of test compounds on the biomarker, serum RBP4 level that is directly linked with formation of toxic lipofuscin fluorophores in the retina.

### Example 6

### Additional TTR Ligands

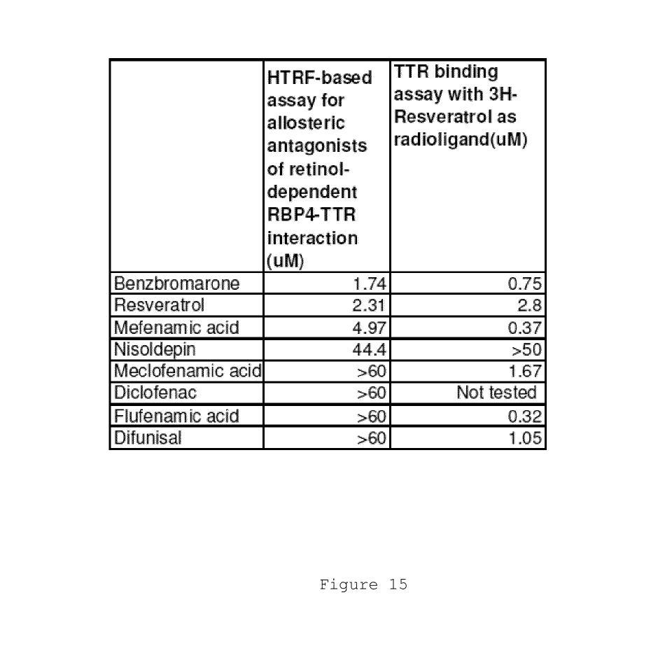

Additional compounds known to be TTR ligands from the literature were tested in the HTRF-based assay for allosteric antagonists of retinol-dependent RBP4-TTR interaction and in TTR binding assay with 3H-resveratrol used as a radioligand. The activity of these compounds is summarized in FIG. 15. Note that benzbromarone, resveratrol and mefenamic acid are capable of antagonizing the retinol-dependent RBP-TTR interaction.

The ligands described herein are a representative list of TTR ligands. Other TTR ligands are known in the art. Some TTR ligands, including their structure and synthesis thereof, are contained within the subsequent listed references. The references listed herein are only a partial list of references describing known TTR ligands.

### Example 7

### Characterization of Tafamidis in the TR-FRET Assay for Allosteric Antagonists of Retinol-Induced RBP4-TTR Interaction

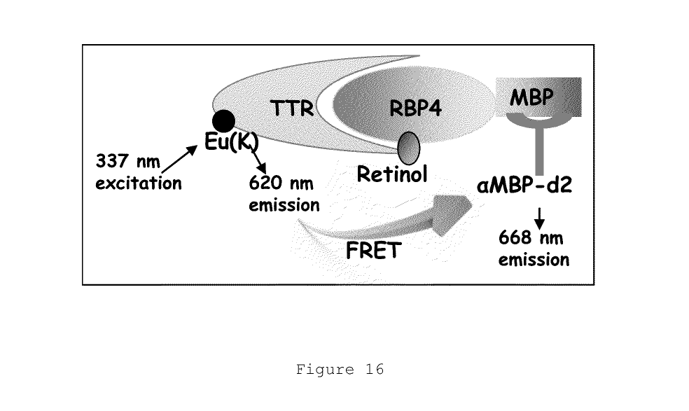

TR-FRET (Time-Resolved Fluorescence Resonance Energy Transfer) is an assay format widely used in characterization of compounds affecting protein-protein interactions [57-59]. HTRF (Homogeneous Time-Resolved Fluorescence) variant of TR-FRET is most advanced as it has improved light capturing due to the use of Eu³⁺ cryptates. In the presence of retinol, RBP4-TTR interaction induces FRET that can be registered as increased ratio of 668/620 fluorescence signals (FIG. 16).

TR-FRET assay for testing the activity of compounds antagonizing the retinol-induced RBP4-TTR interaction has been previously described [60]. This assay was originally developed for characterization of direct RBP4 antagonists that displace retinol from RBP4 [60]. However, if the assay is conducted at saturating retinol concentrations, the screen will allow preferential identification of allosteric antagonists of retinol-dependent RBP4-TTR interaction. Allosteric antagonists are defined as compounds disrupting retinol-dependent RBP4-TTR interaction without displacing retinol from its binding pocket in RBP4. Synthetic TTR ligands are primary candidates for being such allosteric antagonists as the TTR tetramer in a retinol-RBP4-TTR complex contains two unoccupied well-defined ligand binding pockets for thyroxine located in the proximity to the RBP4-TTR interaction interface. Binding of a desired TTR ligands would disrupt RBP4-TTR interaction induced by saturating concentrations of retinol which registered as a decrease in FRET signal (FIG. 16). An assay using E. coli-expressed MBP-tagged RBP4 and commercially available TTR labeled directly with Eu³⁺ cryptate was developed. In addition to MBP-RBP4 and Eu³⁺ (K)-TTR, a detector reagent anti-MBP-d2 was present in the reaction mix. The assay was first optimized in the agonist mode; sensitivity and dynamic range of the assay was optimized in respect to RBP4, TTR and detection reagent concentrations. In order to determine the optimum concentration of all-trans retinol stimulating the RBP4-TTR interaction δ 12-point retinol titration (FIG. 6) was performed. It was demonstrated that all-trans retinol stimulates RBP4-TTR interaction in a dose dependent manner (FIG. 6) with EC₅₀ of 308 nM.

Given that there were no known TTR ligands capable of antagonizing retinol-dependent RBP4-TTR interaction at that time, no TTR-specific positive control could be used in assay development. At the same time, a highly potent RBP4 antagonist, A1120, capable of disrupting retinol-dependent RBP4-TTR interaction with Kᵢ of 8.3 nM has been recently described [61]. The assay was converted to the antagonist mode by testing concentrations of retinol within the 1-10 μM range and using 40 μM concentration of A1120. During conversion of the assay to the antagonist mode significant consideration has been given to the range of appropriate agonist (retinol) concentrations. Retinol concentration has to be high enough in order to allow preferential identification of allosteric antagonists acting independent of retinol binding to RBP4. At the same time, it was desired to use A1120, a direct retinol antagonist, as a positive control in the assay characterization. The compromise retinol concentration in the antagonist mode in regard of the assay sensitivity and dynamic range was found to be in the 4.5-6.5 μM range which is appropriately higher than EC₅₀=308 nM for retinol (FIG. 6). Overall, the antagonist format of the assay was optimized for low-volume 384-well plates with the final volume of 16 uL. Because of the homogenous nature of HTRF assays, no reagent changes or washes were required; there are 2 dispensing steps required to perform this assay. To additionally document assay performance in the antagonist mode, titrations of A1120 and fenretinide, two direct retinol antagonists, were conducted in the presence of high concentrations of retinol (FIG. 7).

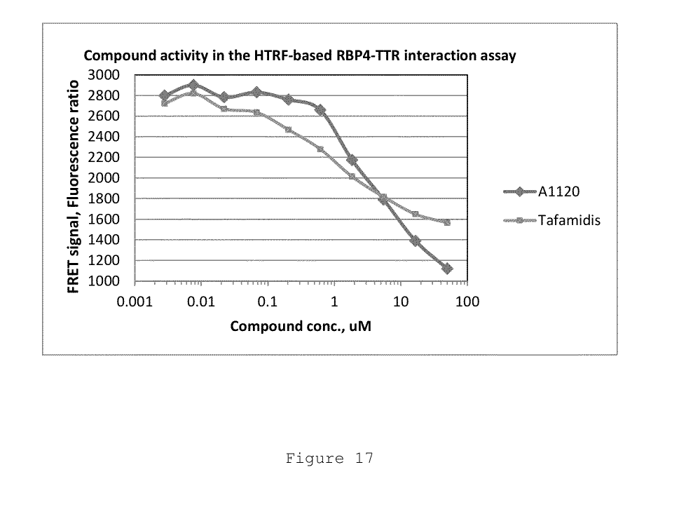

As expected, due to high concentration of retinol required for preferential identification of allosteric inhibitors of retinol-dependent RBP4-TTR interaction, high concentrations of direct retinol antagonists, A1120 and fenretinide, were required for inhibition of the RBP4-TTR interaction in assay conditions (IC₅₀=2.2 μM for A1120 and 17.3 μM for fenretinide). These results confirmed that A1120 can be used as a positive control during assay implementation. Dose-titration of tafamidis was conducted in the HTRF RBP4-TTR interaction assay along with the positive control, A1120 (FIG. 17).

These results established that tafamidis can antagonize retinol-dependent RBP4-TTR interaction. Activity of tafamidis in this assay was compared with the activities of other TTR ligands (Table 1).

TABLE 1
Comparison of tafamidis activity with that of other
TTR ligands in the HTRF-based RBP4-TTR interaction assay.

| | | IC50, |
|---|---|---|
| Cpd | Structure | μM |
| Benz- bromarone | 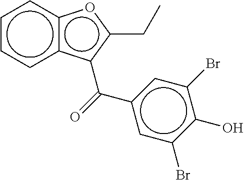 | 0.8 |
| Resveratrol | 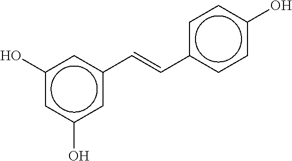 | 1.6 |
| Mefenamic acid | 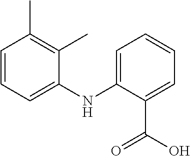 [SMILES: Cc1cccc(Nc2ccccc2C(=O)O)c1C] | 2.1 |
| Tafamidis | 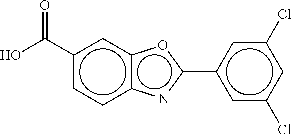 | 1.5 |

### Example 8

### Transthyretin Fluorescence Polarization Binding Assay

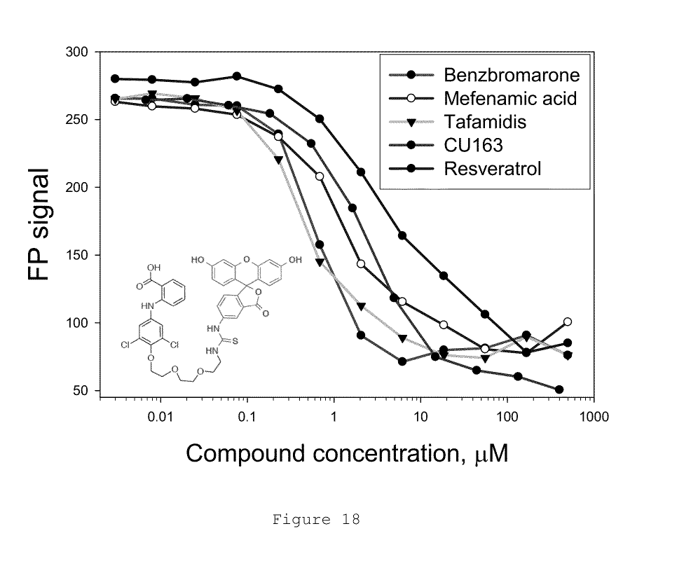

Transthyretin is a tetrameric protein with two clearly defined thyroxine-binding pockets [62]. Numerous publications report the design of the competition binding assays for TTR that utilize [¹²⁵I]-thyroxine as a radioligand [63-65]. Additionally, a synthesis of the FITC-modified TTR ligand that can be used in a fluorescence-polarization-(FP)-based binding assay has recently been reported [66]. For an FP assay implementation, a TTR-FP probe (FIG. 18, inset) was synthesized following a previously described synthetic route [66].

The binding assay conditions were optimized in a 96-well format; the assay demonstrated a 3-6 fold window with a Z score of higher than 0.7. As shown in Table 2, four compounds were definitively shown to bind to TTR as can be judged by the displacement of the FP probe from TTR. While mefenamic acid, tafamidis and resveratrol are well-known TTR ligands [67-68], benzbromarone was not previously known to be a TTR ligand.

In vitro potency data in the TTR binding assay for tafamidis and other compounds are listed in Table 2. The TTR binding potency for benzbromarone, IC₅₀=0.51 μM, is in line with the TTR binding potency of tafamidis, a compound that resulted from a lengthy optimization process [69].

TABLE 2
Compound potency in the FP-TTR binding assay.

| | IC50, |
|---|---|
| Cpd | μM |
| Benzbromarone | 0.51 |
| Mefenamic acid | 1.2 |
| Resveratrol | 4.7 |
| Tafamidis | 0.41 |

### Example 9

### Tafamidis Induces RBP4 Reduction In Vivo Proving Engagement of the Target and Expected Mechanism of Action

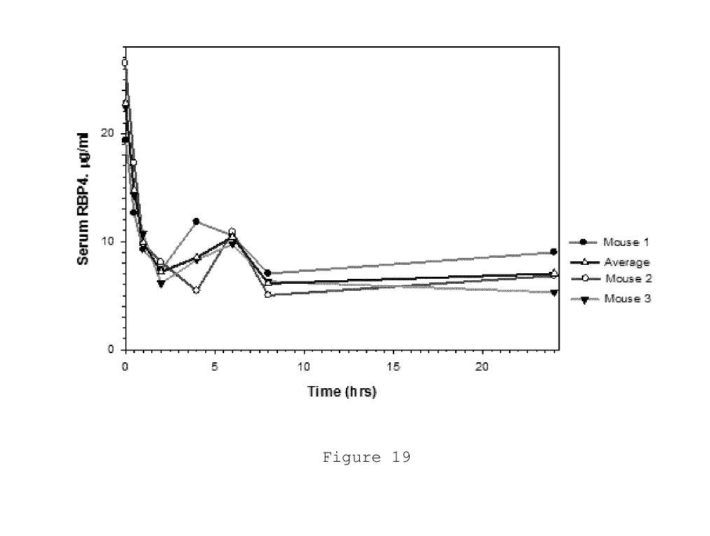

To establish proof of in vivo activity the effect of tafamidis dosing in mice levels of serum RBP4 was studied. Tafamidis was administered through oral gavage at the 50 mg/kg dose. Three mice were used. Blood samples were collected from a tail vein at different timepoints and serum RBP4 was measured using the RBP4 ELISA kit as was previously described [65]. FIG. 19 shows the extent of the RBP4 reduction induced by a single oral tafamidis dose.

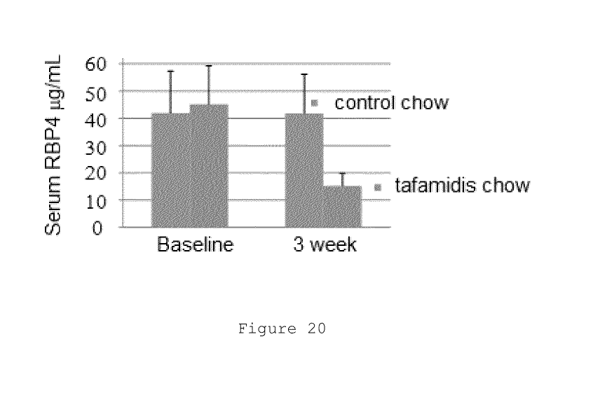

A maximum of 70% decrease in serum RBP4 was induced by tafamidis (FIG. 19). In order to establish that chronic tafamidis dosing can induce the sustained RBP4 reduction, 35 mg/ml tafamidis was orally administered at 35 mg/ml for three weeks to a group of 8 mice. Another group of 8 mice served as control. As shown in FIG. 20, chronic tafamidis administration induced the sustained 67% reduction in serum RBP4.

Given the absolute correlation between RBP4 lowering and reduction in bisretinoid accumulation in the Abca4^{-/-} mouse model that has been established for direct antagonists of the RBP4-TTR interaction from different structural classes [63, 65], tafamidis, benzbromarone and their analogs show the desired efficacy in the Abca4^{-/-} preclinical model of enhanced

### Example 10

### TTR Ligand for Treating Diabetes and Obesity

Diabetes mellitus (diabetes) is a complex chronic disease characterized by elevated levels of blood glucose due to defects in insulin secretion and/or insulin action. To function properly, the human body must have a balanced production of insulin from the pancreas to transport glucose efficiently to other organs and tissues for storage. Any insulin imbalance or loss of sensitivity may cause a chronic overabundance of glucose eventually leading to diabetes (see Wass, J. & Stewart P. M. Oxford Textbook of Endocrinology and Diabetes (2011), the contents of which is hereby incorporated by reference).

Of those individuals with type II diabetes, about 80-90 percent are also diagnosed as obese. Weight gain is common in people who take insulin to treat diabetes. Accordingly, treatment of diabetes is linked to the reduction of obesity as well (see Weir, G. C. Endocrinology Adult and Pediatric: Diabetes Mellitus and Obesity, 6e (2013), the contents of which is hereby incorporated by reference).

RBP4 is an adipocyte-derived ‘signal’ that may contribute to the pathogenesis of type 2 diabetes [70]. Accordingly, the lowering RBP4 by the present allosteric antagonist of the retinol-binding dependent RBP4-TTR interaction is a new strategy for treating type 2 diabetes. Since diabetes is linked to increased obesity, the antagonists of the present application are also useful for treating obesity.

An amount of benzbromarone, resveratrol, mefenamic acid, tafamidis, flufenamic acid, diflunisal, diclofenac or flurbiprofen is administered to a subject afflicted with diabetes. The amount of the compound is effective to treat the subject.

An amount of benzbromarone, resveratrol, mefenamic acid, tafamidis, flufenamic acid, diflunisal, diclofenac or flurbiprofen is administered to a subject afflicted with obesity. The amount of the compound is effective to treat the subject.

### Discussion

Age-related macular degeneration (AMD) is the leading cause of blindness in developed countries. Its prevalence is higher than that of Alzheimer's disease. There is no treatment for the most common dry form of AMD. Dry AMD is triggered by abnormalities in the retinal pigment epithelium (RPE) that lies beneath the photoreceptor cells and provides critical metabolic support to these light-sensing cells. RPE dysfunction induces secondary degeneration of photoreceptors in the central part of the retina called macula. Experimental data indicate that high levels of lipofuscin induce degeneration of RPE and the adjacent photoreceptors in atrophic AMD retinas. In addition to AMD, dramatic accumulation of lipofuscin is the hallmark of Stargardt disease (STGD), an inherited form of juvenile-onset macular degeneration. Other retinal diseases such as Best disease, autosomal-dominant Stargardt-like macular degeneration and others are characterized by excessive lipofuscin accumulation in the retina.

The major cytotoxic component of RPE lipofuscin is a pyridinium bisretinoid A2E. A2E formation occurs in the retina in a non-enzymatic manner and can be considered a by-product of a properly functioning visual cycle. Given the established cytotoxic affects of A2E on RPE and photoreceptors, inhibition of A2E formation could lead to delay in visual loss in patients with dry AMD, STGD, and other retinal disease characterized by excessive lipofuscin accumulation. It was suggested that small molecule visual cycle inhibitors may reduce the formation of A2E in the retina and prolong RPE and photoreceptor survival in patients with dry AMD, STGD, and other retinal disease characterized by excessive lipofuscin accumulation. Rates of the visual cycle and A2E production in the retina depend on the influx of all-trans retinol from serum to the RPE. Pharmacological downregulation of serum retinol is a valid treatment strategy for dry AMD, STGD, and other retinal disease characterized by excessive lipofuscin accumulation. Serum retinol is maintained in circulation as a tertiary complex with retinol-binding protein (RBP4) and transthyretin (TTR). Without interacting with TTR, the RBP4-retinol complex is rapidly cleared due to glomerular filtration. Retinol binding to RBP4 is required for formation of the RBP4-TTR complex; apo-RBP4 does not interact with TTR. Until the present invention, only one class of compounds, competitive antagonists of retinol binding to RBP4, was currently known to block retinol-dependent RBP4-TTR interaction and reduce A2E production in the animal model of excessive lipofuscin accumulation. It was hypothesized that TTR ligands were capable of antagonizing retinol-dependent RBP4-TTR interaction. Before the present invention, TTR had never been considered as a drug target for pharmacological inhibition of the visual cycle or as a drug target for treatment of macular degeneration.

### Serum Transthyretin as a Novel Drug Target for Pharmacological Inhibition of the Visual Cycle

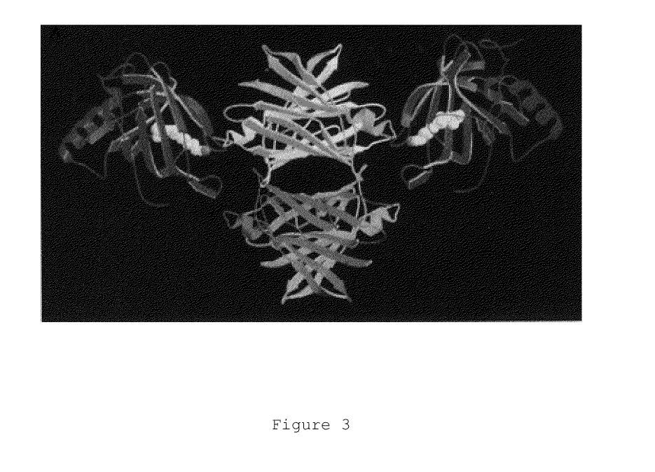

Serum retinol is bound to retinol-binding protein (RBP4) and maintained in circulation as a tertiary complex with RBP4 and transthyretin (TTR)—FIG. 3. Without interacting with TTR, the RBP4-retinol complex is rapidly cleared from circulation due to glomerular filtration. Additionally, formation of the RBP4-TTR-retinol complex is required for receptor-mediated all-trans retinol uptake from serum to the retina.

Disruption of the RBP4-TTR complex leading to fast clearance of low molecular weight RBP4 through glomerular filtration is an established approach to reducing serum retinol level with following inhibition of the visual cycle [13, 16]. Only one class of compounds, competitive antagonists of retinol binding to RBP4, is currently known to block retinol-dependent RBP4-TTR interaction [13, 16]. Two members of this class, fenretinide and A1120, are described in the literature [13, 16]. Both compounds are shown to displace all-trans retinol from RBP4, disrupt the RBP4-TTR interaction, and reduce serum retinol [13, 16, 17].

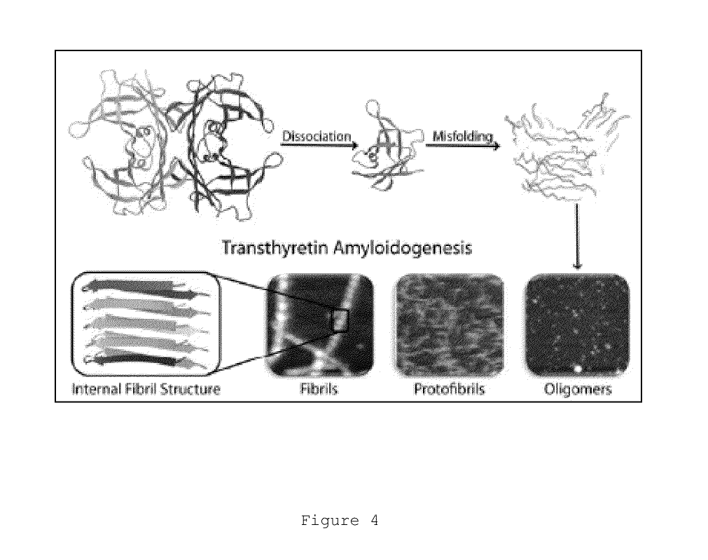

Additionally, fenretinide administration was shown to inhibit the visual cycle and reduce A2E production in the animal model of excessive lipofuscin accumulation [13]. While fenretinide is unlikely to become a treatment for AMD and STGD due to significant safety liabilities associated with its off-target pro-apoptotic and theratogenic activities [18-23], A1120 or its derivatives may potentially become a therapy for the majority of patients with dry AMD and Stargardt disease. However, chronic use of RBP4 antagonists in a sub-population of patients with pro-amyloidogenic mutations in the TTR gene may have unwanted consequences. As illustrated in FIG. 4, in patients with TTR mutations a normally stable TTR tetramer may dissociate into monomers that can partially unfold and misassemble into amyloid fibrils forming pathogenic deposits in the heart and peripheral nerves and causing familial amyloid cardiomyopathy and familial amyloid polyneuropathy [24, 25].

It is known that over 50% of plasma TTR is associated with retinol-RBP4 [27]. TTR knock-out mice are phenotypically normal despite extremely low plasma retinol and RBP4 levels (6% of wild type) [28]. Formation of the tertiary retinol-RBP4-TTR complex stabilizes TTR tetramers and prevents formation of TTR amyloid fibrils [15, 27]. It was reported that the majority of TTR in circulation, including TTR in a complex with holoRBP4, is unliganded since in humans 99% of TTR's natural ligand, thyroxine, is transported by another serum carrier protein, thyroxine-binding globulin [15, 29]. The release of unliganded TTR induced by RBP4 antagonists may facilitate amyloid formation in vulnerable patients with pro-amyloidogenic TTR mutations. It is known that synthetic and endogenous TTR ligands are capable of stabilizing TTR tetramers thus preventing its dissociation into monomers and inhibiting the formation of amyloid fibrils [24, 30, 31].

Based on our data, there are TTR ligands that allosterically antagonize retinol-dependent RBP4-TTR interaction. Such ligands would induce the disruption of the retinol-RBP4-TTR complex with subsequent reduction in serum RBP4 and retinol levels. This would lead to the reduced uptake of retinol to the retina, inhibition of the visual cycle and reduction in formation of cytotoxic A2E. At the same time, such TTR ligands could stabilize TTR tetramers released from the retinol-RBP4-TTR complex preventing the formation of amyloid fibrils in patients who, in addition to dry AMD and STGD, may carry proamyloidogenic mutations in the TTR gene.

Before the present invention, it was not known that TTR ligands can inhibit retinol-dependent RBP4-TTR interaction. Allosteric antagonists of retinol-dependent RBP4-TTR interaction are compounds capable of inhibiting this interaction without binding to the retinal-binding pocket in the RBP4, thus they are not antagonists of retinol binding to the RBP4. Ligand-binding site in TTR is a primary place for binding of allosteric antagonists of retinol-dependent RBP4-TTR interaction. To identify such compounds, an HTRF assay assessing retinol-dependent RBP4-TTR interaction was developed. Importantly this assay was run in the presence of high saturating concentration of retinol. These conditions allowed for the rejection of compound directly competing with retinol for its binding site in RBP4 while favoring compounds inhibiting the interaction allosterically. Two potential allosteric antagonists were identified, benzbromarone and resveratrol which were assessed in a battery of in vitro and in vivo assays.

The data for these compounds may be summarized as follows: (1) the two compounds dose-dependently inhibit RBP-TTR interaction at high retinal concentration, (2) the two compounds do not bind to RBP4, (3) the two compounds bind to TTR, and (4) the two compounds reduce serum RBP4 level when dosed in mice.

Mutations within TTR are responsible for orphan inherited conditions such as familial amyloid cardiomyopathy and familial amyloid polyneuropathy. Tafamidis is a TTR ligand and it is approved in Europe for treatment of familial amyloid polyneuropathy.

Several other known TTR ligands were assessed in the in vitro assays with one compound, mefenamic acid, showing significant activity while others (e.g., Flufenamic acid and Diflunisal) being much weaker antagonists of retinol-dependent RBP4-TTR interaction or not showing the activity at all. The ligands described herein are a representative list of TTR ligands. Other TTR ligands are expected to act analogously to benzbromarone and resveratrol.

Tafamidis acts as an allosteric antagonist of the retinol-dependent RBP4-TTR interaction that can disrupt this interaction without displacing retinol from retinol-binding pocket of RBP4. Due to this antagonistic activity tafamidis may induce serum RBP4 reduction that would lead to the diminished uptake of serum retinol to the retina and inhibition of the formation of cytotoxic bisretinoids. This would suggest that tafamidis may be used as a treatment for age-related macular degeneration, Stargardt's disease, Best's disease and other retinal conditions characterized by the excessive accumulation of lipofuscin. Tafamidis, as well as other TTR ligands, may be used for treatment of ocular conditions which are not characterized by the increased accumulation of lipofuscin but in which modulation of the visual cycle or reduction in the level of visual cycle retinoids may be beneficial. Such conditions may include diabetic retinopathy, light-induced photoreceptor degeneration, and retinal detachment. Tafamidis may also be used for treating non-ophthalmic conditions, such as diabetes and obesity, in which downregulation of RBP4 may be beneficial.

### REFERENCES

1. Petrukhin K: New therapeutic targets in atrophic age-related macular degeneration. Expert Opin Ther Targets 2007, 11(5):625-639.
2. Delori F C, Goger D G, Dorey C K: Age-related accumulation and spatial distribution of lipofuscin in RPE of normal subjects. Investigative ophthalmology & visual science 2001, 42(8):1855-1866.
3. Delori F C: RPE lipofuscin in ageing and age-related macular degeneration. In: Retinal Pigment Epithelium and Macular Disease (Documenta Ophthalmologica). Edited by G. Coscas FOP, vol. 62. Dordrecht, The Netherlands Kluwer Academic Publishers; 1995: 37-45.
4. Dorey C K, Wu G, Ebenstein D, Garsd A, Waiter J J: Cell loss in the aging retina. Relationship to lipofuscin accumulation and macular degeneration. Investigative ophthalmology & visual science 1989, 30(8):1691-1699.
5. Feeney-Burns L, Hilderbrand E S, Eldridge S: Aging human RPE: morphometric analysis of macular, equatorial, and peripheral cells. Investigative ophthalmology & visual science 1984, 25(2):195-200.
6. Holz F G, Bellman C, Staudt S, Schutt F, Volcker H E: Fundus autofluorescence and development of geographic atrophy in age-related macular degeneration. Investigative ophthalmology & visual science 2001, 42(5):1051-1056.
7. von Ruckmann A, Fitzke F W, Bird A C: Fundus autofluorescence in age-related macular disease imaged with a laser scanning ophthalmoscope. Investigative ophthalmology & visual science 1997, 38(2):478-486.
8. Sparrow J R, Fishkin N, Zhou J, Cai B, Jang Y P, Krane S, Itagaki Y, Nakanishi K: A2E, a byproduct of the visual cycle. Vision research 2003, 43(28):2983-2990.
9. Gollapalli D R, Rando R R: The specific binding of retinoic acid to RPE65 and approaches to the treatment of macular degeneration. Proceedings of the National Academy of Sciences of the United States of America 2004, 101(27):10030-10035.
10. Maiti P, Kong J, Kim S R, Sparrow J R, Allikmets R, Rando R R: Small molecule RPE65 antagonists limit the visual cycle and prevent lipofuscin formation. Biochemistry 2006, 45(3):852-860.
11. Radu R A, Mata N L, Nusinowitz S, Liu X, Sieving P A, Travis G H: Treatment with isotretinoin inhibits lipofuscin accumulation in a mouse model of recessive Stargardt's macular degeneration. Proceedings of the National Academy of Sciences of the United States of America 2003, 100(8):4742-4747.
12. Radu R A, Mata N L, Nusinowitz S, Liu X, Travis G H: Isotretinoin treatment inhibits lipofuscin accumulation in a mouse model of recessive Stargardt's macular degeneration. Novartis Found Symp 2004, 255:51-63; discussion 63-57, 177-178.
13. Radu R A, Han Y, Bui T V, Nusinowitz S, Bok D, Lichter J, Widder K, Travis G H, Mata N L: Reductions in serum vitamin A arrest accumulation of toxic retinal fluorophores: a potential therapy for treatment of lipofuscin-based retinal diseases. Investigative ophthalmology & visual science 2005, 46(12):4393-4401.
14. Monaco H L, Rizzi M, Coda A: Structure of a complex of two plasma proteins: transthyretin and retinol-binding protein. Science 1995, 268(5213):1039-1041.
15. White J T, Kelly J W: Support for the multigenic hypothesis of amyloidosis: the binding stoichiometry of retinol-binding protein, vitamin A, and thyroid hormone influences transthyretin amyloidogenicity in vitro. Proc Natl Acad Sci USA 2001, 98(23):13019-13024.
16. Motani A, Wang Z, Conn M, Siegler K, Zhang Y, Liu Q, Johnstone S, Xu H, Thibault S, Wang Y et al: Identification and characterization of a non-retinoid ligand for retinol-binding protein 4 which lowers serum retinol-binding protein 4 levels in vivo. J Biol Chem 2009, 284(12):7673-7680.
17. Berni R, Formelli F: In vitro interaction of fenretinide with plasma retinol-binding protein and its functional consequences. FEBS Lett 1992, 308(1):43-45.
18. Adams W R, Smith J E, Green M H: Effects of N-(4-hydroxyphenyl)retinamide on vitamin A metabolism in rats. Proc Soc Exp Biol Med 1995, 208(2):178-185.
19. Puduvalli V K, Saito Y, Xu R, Kouraklis G P, Levin V A, Kyritsis A P: Fenretinide activates caspases and induces apoptosis in gliomas. Clin Cancer Res 1999, 5(8):2230-2235.
20. Holmes W F, Soprano D R, Soprano K J: Synthetic retinoids as inducers of apoptosis in ovarian carcinoma cell lines. J Cell Physiol 2004, 199(3):317-329.
21. Simeone A-M, Ekmekcioglu S, Broemeling L D, Grimm E A, Tari A M: A novel mechanism by which N-(4-hydroxyphenyl)retinamide inhibits breast cancer cell growth: the production of nitric oxide. Mol Cancer Ther 2002, 1(12):1009-1017.
22. Fontana J A, Rishi A K: Classical and novel retinoids: their targets in cancer therapy. Leukemia 2002, 16(4):463-472.
23. Samuel W, Kutty R K, Nagineni S, Vijayasarathy C, Chandraratna R A S, Wiggert B: N-(4-hydroxyphenyl)retinamide induces apoptosis in human retinal pigment epithelial cells: retinoic acid receptors regulate apoptosis, reactive oxygen species generation, and the expression of heme oxygenase-1 and Gadd153. J Cell Physiol 2006, 209(3):854-865.
24. Klabunde T, Petrassi H M, Oza V B, Raman P, Kelly J W, Sacchettini J C: Rational design of potent human transthyretin amyloid disease inhibitors. Nat Struct Biol 2000, 7(4):312-321.
25. Sacchettini J C, Kelly J W: Therapeutic strategies for human amyloid diseases. Nat Rev Drug Discov 2002, 1(4):267-275.
26. Connelly S, Choi S, Johnson S M, Kelly J W, Wilson I A: Structure-based design of kinetic stabilizers that ameliorate the transthyretin amyloidoses. Curr Opin Struct Biol 2010, 20(1):54-62.
27. Hyung S J, Deroo S, Robinson C V: Retinol and retinol-binding protein stabilize transthyretin via formation of retinol transport complex. ACS Chem Biol 2010, 5(12):1137-1146.
28. Wolf G: Retinol transport and metabolism in transthyretin-“knockout” mice. Nutr Rev 1995, 53(4 Pt 1):98-99.
29. Liz M A, Mar F M, Franquinho F, Sousa M M: Aboard transthyretin: From transport to cleavage. IUBMB Life 2010, 62(6):429-435.
30. Miroy G J, Lai Z, Lashuel H A, Peterson S A, Strang C, Kelly J W: Inhibiting transthyretin amyloid fibril formation via protein stabilization. Proc Natl Acad Sci USA 1996, 93(26):15051-15056.
31. Alhamadsheh M M, Connelly S, Cho A, Reixach N, Powers E T, Pan D W, Wilson I A, Kelly J W. Graef I A: Potent kinetic stabilizers that prevent transthyretin-mediated cardiomyocyte proteotoxicity. Sci Transl Med 2011, 3(97):97ra81.
32. Glickman J F, Wu X, Mercuri R, Illy C, Bowen B R, He Y, Sills M: A comparison of ALPHAScreen, TR-FRET, and TRF as assay methods for FXR nuclear receptors. J Biomol Screen 2002, 7(1):3-10.
33. Fujimura T, Sakuma H, Ohkubo-Suzuki A, Aramori I, Mutoh S: Unique properties of coactivator recruitment caused by differential binding of FK614, an anti-diabetic agent, to peroxisome proliferator-activated receptor gamma. Biol Pharm Bull 2006, 29(3):423-429.
34. Zhou G, Cummings R, Li Y, Mitra S, Wilkinson H A, Elbrecht A, Hermes J D, Schaeffer J M, Smith R G, Moller D E: Nuclear receptors have distinct affinities for coactivators: characterization by fluorescence resonance energy transfer. Mol Endocrinol 1998, 12(10):1594-1604.
35. Cogan U, Kopelman M, Mokady S, Shinitzky M: Binding affinities of retinol and related compounds to retinol binding proteins. European journal of biochemistry/FEBS 1976, 65(1):71-78.
36. Hamilton J A, Benson M D: Transthyretin: a review from a structural perspective. Cell Mol Life Sci 2001, 58(10):1491-1521.
37. Lens M C, Spiertz C, Brouwer A, Koeman J H: Different competition of thyroxine binding to transthyretin and thyroxine-binding globulin by hydroxy-PCBs, PCDDs and PCDEs. Eur J Pharmacol 1994, 270(2-3):129-136.
38. Chang L, Munro S L, Richardson S J, Schreiber G: Evolution of thyroid hormone binding by transthyretins in birds and mammals. Eur J Biochem 1999, 259(1-2):534-542.
39. Radovic B, Mentrup B, Kohrle J: Genistein and other soya isoflavones are potent ligands for transthyretin in serum and cerebrospinal fluid. Br J Nutr 2006, 95(6):1171-1176.
40. Bourgault S, Choi S, Buxbaum J N, Kelly J W, Price J L, Reixach N: Mechanisms of transthyretin cardiomyocyte toxicity inhibition by resveratrol analogs. Biochem Biophys Res Commun 2011, 410(4):707-713.
41. Raghu P, Reddy G B, Sivakumar B: Inhibition of transthyretin amyloid fibril formation by 2,4-dinitrophenol through tetramer stabilization. Arch Biochem Biophys 2002, 400(1):43-47.
42. Baures P W, Peterson S A, Kelly J W: Discovering transthyretin amyloid fibril inhibitors by limited screening. Bioorg Med Chem 1998, 6(8):1389-1401.
43. Lee J W, Lee Y C, Na S Y, Jung D J, Lee S K: Transcriptional coregulators of the nuclear receptor superfamily: coactivators and corepressors. Cell Mol Life Sci 2001, 58(2):289-297.
44. Savkur R S, Burris T P: The coactivator LXXLL nuclear receptor recognition motif. J Pept Res 2004, 63(3):207-212.
45. Chen J D, Evans R M: A transcriptional co-repressor that interacts with nuclear hormone receptors. Nature 1995, 377(6548):454-457.
46. Morais-de-Sa, E. et al.: The Crystal Structure of transthyretin in Complex with Diethylstilbestrol. The Journal of Biological Chemistry 2004, 279, 53483-53490.
47. Almeida M R, Gales L, Damas A M, Cardoso I, Saraiva M J: Small transthyretin (TTR) ligands as possible therapeutic agents in TTR amyloidoses. Curr Drug Targets CNS Neurol Disord. 2005, 4(5), 587-96.
48. Arsequell G, Planas A.: Methods to Evaluate the Inhibition of TTR Fibrillogenesis Induced by Small Ligands. Curr Med. Chem. 2012, 19(15), 2343-55.
49. Kolstoe, S. E. et al.: Trapping of palindromic ligands within native transthyretin prevents amyloid formation. Proceedings of the National Academy of Sciences (PNAS) 2010, vol. 107, no. 47, 20483-20488.
50. Julius, R. L.; Hawthorne M. F: Amyloid disease prevention by transthyretin native state complexation with carborane derivatives lacking cyclooxygenase inhibition. Drug News Perspect. 2008, 21(5), 258-66.
51. Richardson, S. J.; Cody, V.: Recent Advances in Transthyretin Evolution, Structure and Biological Functions. (Springer, 2009).
52. Oza, V. B. et al.: Synthesis and evaluation of anthranilic acid-based transthyretin amyloid fibril inhibitors. Bioorganic & Medicinal Chemistry Letters, 1999, Vol 9, 1-6.
53. Baures, P. W. et al.: Discovering transthyretin amyloid fibril inhibitors by limited screening. Bioorganic & Medicinal Chemistry, 1998, Vol 6, 1389-1401.
54. Baures, P. W. et al.: Synthesis and evaluation of inhibitors of transthyretin amyloid formation based on the non-steroidal anti-inflammatory drug, flufenamic acid. Bioorganic & Medicinal Chemistry, 1999, Vol 7, Issue 7, 1339-1347.
55. de Lartigue J. Tafamidis for Transthyretin Amyloidosis. Drug Today. May 2012; 48(5):331-337.
56. de Carvalho M. Is it better than it seems or just good enough? The tafamidis saga. Muscle Nerve. 2012; 46(6):839-840.
57. Glickman J F, Wu X, Mercuri R, et al. A comparison of ALPHAScreen, TR-FRET, and TRF as assay methods for FXR nuclear receptors. J Biomol Screen. 2002; 7(1):3-10.
58. Fujimura T, Sakuma H, Ohkubo-Suzuki A, Aramori I, Mutoh S. Unique properties of coactivator recruitment caused by differential binding of FK614, an anti-diabetic agent, to peroxisome proliferator-activated receptor gamma. Biol Pharm Bull. 2006; 29(3):423-429.
59. Zhou G, Cummings R, Li Y, et al. Nuclear receptors have distinct affinities for coactivators: characterization by fluorescence resonance energy transfer. Mol. Endocrinol. 1998; 12(10):1594-1604.
60. Dobri N, Qin Q, Kong J, et al. A1120, a Nonretinoid RBP4 Antagonist, Inhibits Formation of Cytotoxic Bisretinoids in the Animal Model of Enhanced Retinal Lipofuscinogenesis. Investigative ophthalmology & visual science. 2013; 54(1):85-95.
61. Motani A, Wang Z, Conn M, et al. Identification and characterization of a non-retinoid ligand for retinol-binding protein 4 which lowers serum retinol-binding protein 4 levels in vivo. J Biol. Chem. Mar. 20, 2009; 284(12):7673-7680.
62. Hamilton J A, Benson M D. Transthyretin: a review from a structural perspective. Cell Mol Life Sci. 2001; 58(10):1491-1521.
63. Lans M C, Spiertz C, Brouwer A, Koeman J H. Different competition of thyroxine binding to transthyretin and thyroxine-binding globulin by hydroxy-PCBs, PCDDs and PCDFs. Eur J. Pharmacol. Apr. 4, 1994; 270(2-3):129-136.
64. Chang L, Munro S L, Richardson S J, Schreiber G. Evolution of thyroid hormone binding by transthyretins in birds and mammals. Eur J. Biochem. January 1999; 259(1-2):534-542.
65. Radovic B, Mentrup B, Kohrle J. Genistein and other soya isoflavones are potent ligands for transthyretin in serum and cerebrospinal fluid. Br J. Nutr. June 2006; 95(6):1171-1176.
66. Alhamadsheh M M, Connelly S, Cho A, et al. Potent kinetic stabilizers that prevent transthyretin-mediated cardiomyocyte proteotoxicity. Sci Transl Med. 2011; 3(97):97ra81.
67. Munro S L, Lim C F, Hall J G, et al. Drug competition for thyroxine binding to transthyretin (prealbumin): comparison with effects on thyroxine-binding globulin. J Clin Endocrinol Metab. June 1989; 68(6):1141-1147.
68. Sacchettini J C, Kelly J W. Therapeutic strategies for human amyloid diseases. Nat Rev Drug Discov. April 2002; 1(4):267-275.
69. Johnson S M, Connelly S, Fearns C, Powers E T, Kelly J W. The transthyretin amyloidoses: from delineating the molecular mechanism of aggregation linked to pathology to a regulatory-agency-approved drug. J Mol. Biol. Aug. 10, 2012; 421(2-3):185-203.
70. Yang, Q. et al. Serum retinol binding protein 4 contributes to insulin resistance in obesity and type 2 diabetes. Nature. 2005 Jul. 21; 436(7049):356-62.

1. A method for treating a disease characterized by excessive lipofuscin accumulation in the retina of a mammal afflicted therewith comprising administering to the mammal an effective amount of a transthyretin (TTR) ligand.

2. The method of claim 1 wherein the disease is further characterized by bisretinoid-mediated macular degeneration.

3. The method of claim 1, wherein the TTR ligand is an allosteric antagonist of retinol dependent RBP4-TTR interaction.

4. The method of claim 1, wherein the TTR ligand stabilizes the tetrameric structure of TTR.

5. The method of claim 1, wherein the amount of the ligand is effective to lower serum concentration of RBP4 in the mammal.

6. The method of claim 1, wherein the amount of the ligand is effective to lower the retinal concentration of a bisretinoid in lipofuscin in the mammal.

7. The method of claim 2, wherein the bisretinoid is A2E.

8. The method of claim 2, wherein the bisretinoid is isoA2E.

9. The method of claim 2, wherein the bisretinoid is A2-DHP-PE.

10. The method of claim 2, wherein the bisretinoid is atRAL di-PE.

11. The method of claim 1, wherein the disease characterized by excessive lipofuscin accumulation in the retina is Age-Related Macular Degeneration.

12. The method of claim 1, wherein the disease characterized by excessive lipofuscin accumulation in the retina is dry (atrophic) Age-Related Macular Degeneration.

13. The method of claim 1, wherein the disease characterized by excessive lipofuscin accumulation in the retina is Stargardt Disease.

14. The method of claim 1, wherein the disease characterized by excessive lipofuscin accumulation in the retina is Best disease.

15. The method of claim 1, wherein the disease characterized by excessive lipofuscin accumulation in the retina is adult vitelliform maculopathy.

16. The method of claim 1, wherein the disease characterized by excessive lipofuscin accumulation in the retina is Stargardt-like macular dystrophy.

17. The method of claim 1, wherein the TTR ligand is benzbromarone, resveratrol, mefenamic acid, tafamidis, flufenamic acid, diflunisal, diclofenac or flurbiprofen.
